(19) United States
(12) Patent Application Publication
(10) Pub. No.: US 2026/0279522 A1
(43) Pub. Date: Sep. 17, 2026

(54) MEDICAL DEVICE DATA PLATFORM

(71) Applicant: Murj, Inc., Santa Cruz, CA (US)

(72) Inventors: Todd Butka, Santa Cruz, CA (US); Chris Irving, Santa Cruz, CA (US); Megan McCabe, San Francisco, CA (US); Deepak Nadipelly, Boston, MA (US)

(21) Appl. No.: 19/281,379

(22) Filed: Jul. 25, 2025

Related U.S. Application Data
(60) Provisional application No. 63/770,618, filed on Mar. 12, 2025.

(51) Int. Cl. G16H 10/60; G16H 30/40; G16H 40/67

(52) U.S. Cl. CPC G16H 10/60; G16H 30/40; G16H 40/67

(57) ABSTRACT

Example medical device data platforms are disclosed herein. In an example, the platform may include at least one integration device to obtain information originating from a plurality of implantable medical devices manufactured by a plurality of manufacturers and implanted in a plurality of patients. The system may also include an actionability evaluator to process the accessed information and generate provider-oriented information.

### CROSS-REFERENCE TO RELATED APPLICATIONS

The present application claims priority to U.S. Provisional Patent Application No. 63/770,618 filed on Mar. 12, 2025, and entitled “MEDICAL DEVICE DATA PLATFORM.” This application is incorporated by reference in its entirety herein.

### TECHNICAL FIELD

Aspects of the present disclosure generally relate to remote healthcare and more particularly to systems and methods for management of data transmitted by medical devices.

### BACKGROUND

Many medical devices, for example, implantable medical devices, implantable cardiac devices (e.g., cardiac pacemakers, implantable cardioverter defibrillators (ICDs), implantable loop recorders (ILRs), etc.), wearable devices (smartwatches, fitness trackers, etc.) include a wireless communication interface whereby information relating to the operation of the device (e.g., diagnostic information) may be regularly or periodically transmitted outside the body of the patient periodically to a separate computing system or device for analysis. This data can be reviewed and analyzed by medical staff to inform the care of the patient.

With the above aspects in mind, as well as others not explicitly discussed herein, various implementations of a medical device data access system are disclosed herein.

### SUMMARY

In some aspects, the techniques described herein relate to a medical device data platform including: an integration manager configured to obtain a plurality of data transmissions, each data transmission originating from a medical device associated with a patient; and an actionability evaluator determining, for each data transmission, generating provider-oriented information by applying a label to the data transmission, wherein the label is one of an actionable label or a non-actionable label.

In some aspects, the techniques described herein relate to a method including: obtaining a plurality of data transmissions from a plurality of medical devices associated with a plurality of patients; generating provider-oriented information by applying a label to each data transmission of the plurality of data transmissions, wherein the label is one of an actionable label or a non-actionable label; and generating a provider portal displaying the plurality of data transmissions and the applied actionable label associated with each data transmission of the plurality of data transmissions.

While multiple implementations are disclosed, still other implementations of the present disclosure will become apparent to those skilled in the art from the following detailed description, which depicts and describes illustrative examples. As will be realized, the implementations described herein are capable of modifications in various aspects, all without departing from the scope of the present disclosure. Accordingly, the drawings and detailed description are to be regarded as illustrative in nature and not restrictive.

### BRIEF DESCRIPTION

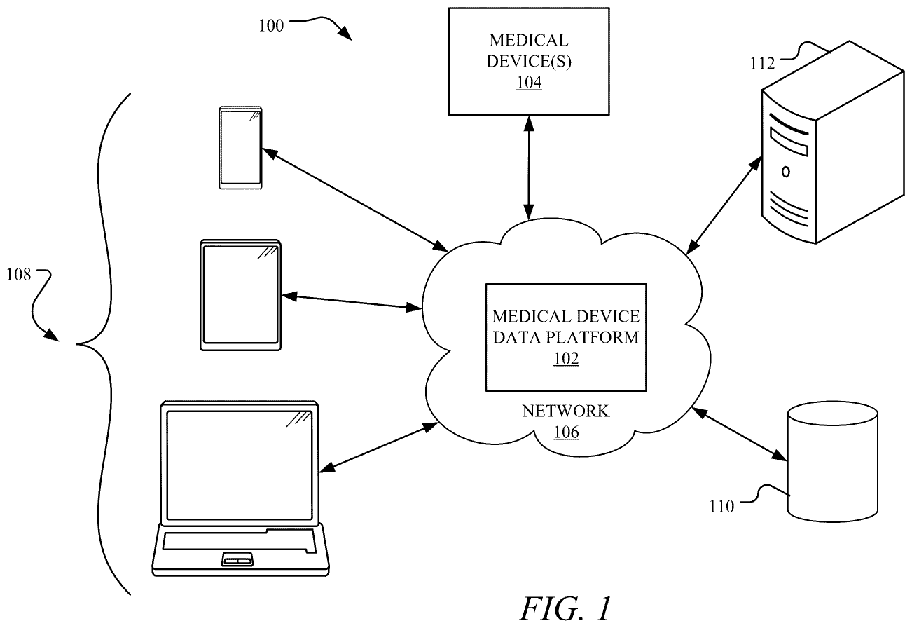

FIG. 1 illustrates an example network environment, including a medical device data platform for managing data received from one or more medical devices.

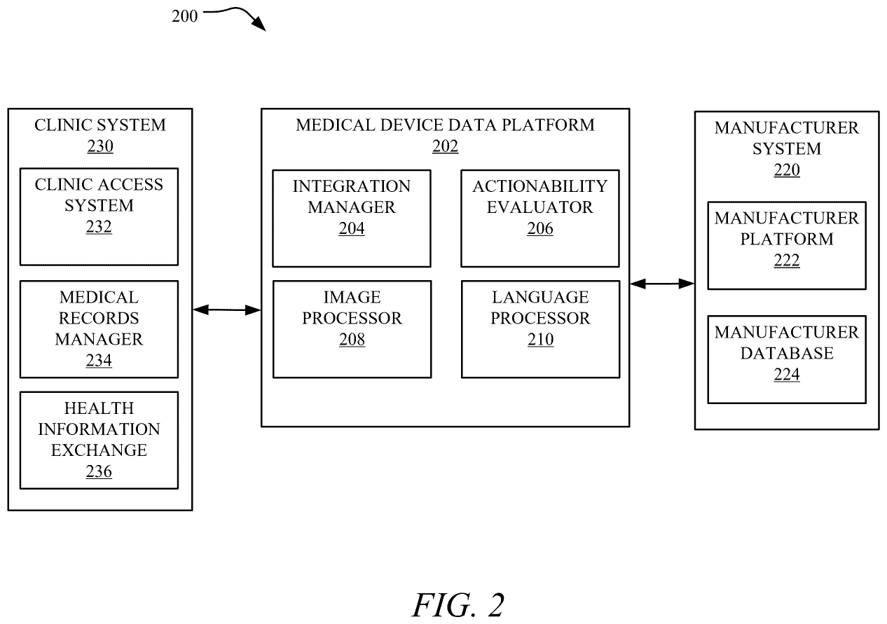

FIG. 2 is a block diagram of a medical device data communication system including an example medical device data platform.

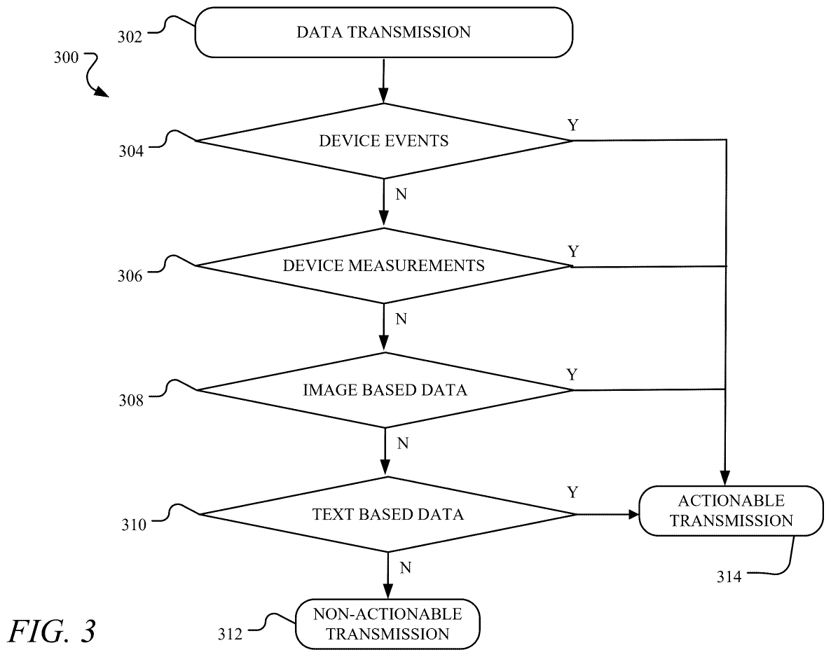

FIG. 3 depicts a flowchart of the analysis of a data transmission.

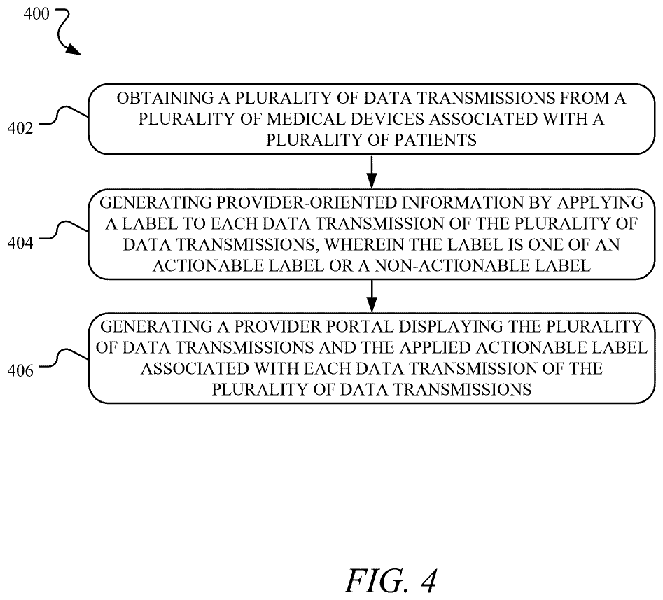

FIG. 4 depicts an example method of providing provider-oriented information based on data transmissions received from a plurality of medical devices.

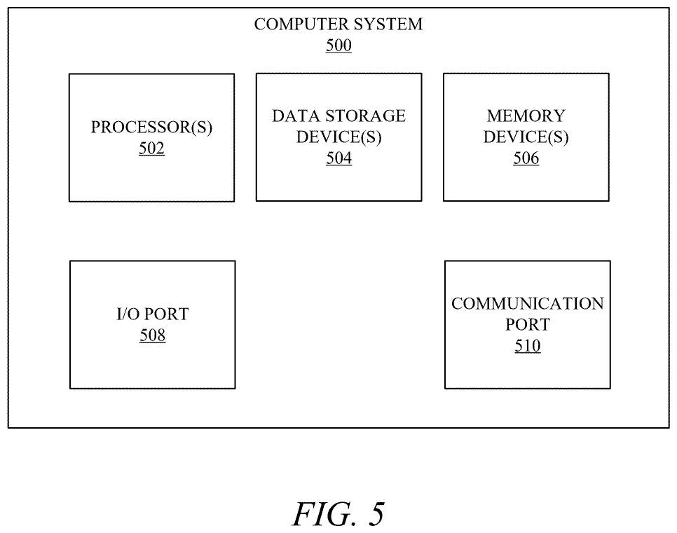

FIG. 5 is a block diagram illustrating an example computing system that may implement various systems and methods discussed herein.

### DETAILED DESCRIPTION

The following detailed description relates to a medical device data platform (or also referenced herein as a platform). In one example, a medical device data platform may include at least one manager configured to obtain data transmissions originating from a plurality of medical devices manufactured by a plurality of manufacturers and associated with a plurality of patients. As described above, the medical devices could include implantable medical devices or wearable devices. The medical device data platform may also include an actionability evaluator configured to process the obtained data transmissions to generate provider-oriented information including labels pertaining to actionability of each of the data transmissions. The label indicates each data transmission as actionable or non-actionable. An actionable label indicates some sort of review or action for a medical care provider. A non-actionable label indicates no immediate action. In other words, data transmissions that are not related to providing patient care is differentiated from data transmissions including information only, so that the medical care provider can focus on providing the urgent attention to the data received from medical devices without compromising the standard of care.

In some implementations described herein, the medical device data platform receives or otherwise obtains data, sometimes called data transmissions, from a medical device implanted in a patient. In some instances, a data manufacturer may have configured the medical device to send data related to everything that is sensed by the implanted medical device. However, not all data may be significant or related to an event that needs urgent attention by a medical care provider. In other words, some data transmitted by the medical device may include non-actionable transmission. Further the medical care provider may be receiving data from many patients, which may complicate the provider's ability to prioritize. Accordingly, the ability to parse data requiring urgent care or an action by the medical care provider may expedite triage of a patient.

Additionally, different medical device manufacturers produce data corresponding their respective product specifications and standards. Accordingly, the solution, as disclosed herein, provides a standardized set of events and their corresponding data across a wide range of medical devices from a plurality of different manufacturers that help the medical care provider to provide the highest quality care to the patient without needing to refer to the details of each medical device's specification or product manuals. Accordingly, the implementations described herein help medical care provider by providing them prioritized and relevant information to care for their patients.

Implementations in the present disclosure identify actionable data transmissions and non-actionable data transmissions and label them accordingly for the medical care provider and thereby facilitate efficient use of the medical care provider's time and resources. In other words, data corresponding to actionable items is highlighted to the medical care provider.

In some implementations, data received from the medical device may be divided or grouped into four categories, where each category may provide a specific type of data to the medical care provider. By way of an example, the four categories may include (i) device events (e.g. device-detected events and/or device-recorded events), (ii) device measurements, (iii) image-based data (e.g. electrogram (EGM) related data and events), and (iv) text-based data (e.g. clinical notes). The data may be received in structured data files or pdfs (for example, as related to items (i)-(iii) described above), or created by clinical users and stored in Murj (for example, item (iv) described above).

A combination of methods, including deterministic, traditional machine learning (ML), and generative AI algorithms, may be employed to assess the actionability of each of the categories of data. If any of the data within a medical device transmission is deemed actionable, then the entire medical device transmission will be categorized as actionable and highlighted to the care provider. Conversely, there is no determination of an actionable item within the data transmission, the data transmission can be categorized as non-actionable. Transmissions can be sorted based upon the assigned labels of actionable or non-actionable, enabling providers to focus efforts on actionable transmissions. At some point, workflow changes may be introduced to bypass steps in the transmission review process when a transmission is classified as non-actionable. As described in the present disclosure, certain types of data or events classified as actionable may also be referred to as actionable criteria.

In some implementations, and by way of a non-limiting example, the actionable criteria may include rules that classify device-detected events, such as arrhythmias or abnormal heart rhythms, as actionable or non-actionable. By way of non-limiting example, the medical device may report an event of ventricular tachycardia lasting 15 seconds, and the rule may be that ventricular tachycardia events lasting more than 10 seconds be classified as actionable. Because the transmission contains data classified as actionable, the transmission will be classified as actionable and displayed to the care provider as such. In addition to the overall transmission actionability status, the data classified as actionable will be highlighted to the provider.

In some implementations, and by way of a non-limiting example, the actionable criteria may include rules that classify device measurements (or device functions), such as battery percent remaining, as actionable or non-actionable. By way of non-limiting example, the transmission may report remaining battery longevity less than 2 months, and the rule may be that battery longevity less than 3 months is classified as actionable. In this case, the transmission will be classified as actionable, and displayed to the care provider as such, with the evidence of low battery highlighted. By way of another non-limiting example, the device may report that the patient had an average lead impedance of 500 ohms over the last 3 months, but only 200 ohms in the last week. The classification rule may state that impedance drops greater than 100 ohms are classified as actionable. In this case, the impedance would be considered actionable data, and the transmission classified as actionable. The transmission actionability status and the evidence for actionability of a large impedance change would be highlighted to the care provider in the platform.

In some implementations, the solution may involve designing prompts for a large language model (LLM) to assign actionable or non-actionable labels to cardiac device transmissions by processing both text and images from PDFs and evaluating the information against a provided care protocol or set of rules. This approach identifies and summarizes actionable conditions, aligns findings with established clinical criteria, and assigns labels of actionable or non-actionable, ultimately generating a concise, actionable summary to support efficient triage on the platform.

In some implementations, and by way of a non-limiting example, the actionable criteria may include rules that classify electrograms (EGM) in the device transmissions as actionable or non-actionable. By way of non-limiting example, there may be a rule stating that evidence of loss of capture (LOC) in an EGM is actionable. Thus, a transmission containing an EGM with evidence of LOC would be classified as actionable and displayed to the care provider as such. Image analysis may be carried out using traditional machine learning methods, such as convolutional neural networks, or using generative AI methods. Image analysis algorithms may also be referenced herein as image classification algorithms.

In some implementations, and by way of a non-limiting example, the actionable criteria may include an analysis of clinical notes. Clinical notes may also include provider-generated notes, suggestions or recommendations for one or more clinical tags based upon user input. The clinical notes may be generated based upon events observed by the device. By way of non-limiting example, there may be a rule stating that battery remaining less than 3 months is actionable, but battery remaining less than 3 months with a clinical note stating that the patient has been scheduled for a device replacement procedure is non-actionable. Text analysis may be carried out using traditional natural language processing (NLP) or generative AI methods. When conditions meet the actionable criteria for the patient, natural language processing (NLP) techniques may be used to analyze any available clinical notes to identify specific events requiring attention of the medical care provider.

In an implementation, the clinician notes can be processed using NLP techniques to categorize transmissions as actionable or non-actionable by analyzing textual content of the clinician notes to identify patterns, keywords and contextual information that indicate the need for action. In one implementation, the processing technique includes preprocessing the notes to clean and standardize text of the clinician notes, applying the NLP technique, such as, for example, tokenization, entity recognition and sentiment analysis to extract meaningful features, to predict whether a transmission requires action using the extracted features.

As described herein, different device manufacturers may have different threshold criteria for different events, and the medical device data platform may filter or transform the received events and data from various implantable medical devices to a standardized format that is actionable by the medical care provider. Additionally, or alternatively, data corresponding to the patient and data corresponding to the specific implantable medical device are also transmitted along with the data received from the implantable medical device. The data may be stored in a database along with a patient identifier so that all data are searchable and available based upon a patient identifier.

In some implementations, the received data from the medical device may also include details about the specific medical device such as device identifier, manufacturer information, model information, etc., which may be then used to collect additional information such as documentation related the medical device from the device manufacturer's website using an application programming interface (API). The documentation thus retrieved from the device manufacturer's website may be stored in a database and used to compare and analyze data received from the device. The documentation retrieved from the device manufacturer's website may include an image and/or text data. The image data may be used to analyze images generated from the image data received from the implantable device using image analysis algorithms such as deep learning algorithms, and/or convolutional neural networks. Text data may be analyzed using natural language processing (NLP) algorithms.

The data is thus labeled so that actionable data is highlighted to an agent assigned to process data for the specific implantable device for further curation. The curated data by an agent experienced with the specific device are then made available to the medical care provider for taking necessary action. The data made available to the medical care provider is supplemented with prior or historical data of the patient so that the medical care provider may determine any improvement or worsening in the patient's health. Severity level may be determined and included in the data to help the medical care provider to determine severity of any improvement or worsening in the patient's health. Additionally, a criticality level may be associated with data corresponding to each event requiring the medical care provider's attention to prioritize the care given to the patient.

In some implementations, filtered out data may be made available to an operator for further verification and modifying or training the algorithms for improving the algorithms to identify and make the data available to the medical care provider without a delay introduced by verification by an operator.

As a result of at least some of the implementations discussed in greater detail below, diagnostics and related information from medical devices manufactured by a plurality of manufacturers, each of which may provide its own proprietary data in a unique format, may be processed to provide information that is tailored to patients and/or care providers (e.g., doctors, nurses, technicians, etc.) regardless of the manufacturer of the particular device. Further, automating the accessing and processing of the data may relieve the manufacturer and/or the care provider of at least some of the data processing and analysis tasks with which they are currently burdened. Other aspects and potential advantages of the implementations disclosed herein are also presented below.

The platform described herein supports a pipeline of medical device data achieved by utilizing the medical device data platform disclosed herein. As described herein, the medical device data platform analyzes and processes data received from the plurality of medical devices along with data received or retrieved from one or more application servers or web servers of a medical device manufacturer.

As described herein, data received or retrieved from the one or more application servers or web servers of the medical device manufacturer may include, but are not limited to only, device operating documents, device specifications, device alerts, high and low measurement values for various measurement parameters, and/or example images of normal and abnormal measurements.

The medical device data platform can be utilized to identify actionable data (also referenced herein as actionable transmission or actionable data transmission) and non-Attorney actionable data (also referenced herein as non-actionable transmission or non-actionable data transmission). Accordingly, the device staff identifies the non-actionable data and filters it out from reaching the medical care provider such as, the physician, so that the medical care provider has not to waste their time and resources to review information that does not help to provide a quality care to the patient. The device staff may include one or more professionals trained for one or more medical devices. Accordingly, the device staff may further filter out data that is not needed to provide emergency care to a patient by the physician. In other words, data corresponding to actionable items is only funneled to the physician.

FIG. 1 illustrates an example network environment 100, including a medical device data platform 102 running on a server 112 or other computing device coupled with a network 106, for managing one or more medical devices 104. As described above, the one or more medical devices 104 may include, but are not limited to, implantable medical devices, implantable cardiac devices (e.g., cardiac pacemakers, implantable cardioverter defibrillators (ICDs), implantable loop recorders (ILRs), etc.), wearable devices (smartwatches, fitness trackers, etc.).

In one implementation, a user, such as a medical care provider, a member of a medical team, and/or a third party to which access is delegated to manage or complete management services, accesses and interacts with a medical device manager portal using a user device 108 to access or manage one or more medical devices via a network 106 (e.g., the Internet). The user device 108 is generally any form of computing device capable of interacting with the network 106, such as a personal computer, terminal, workstation, portable computer, mobile device, smartphone, tablet, multimedia console, etc. The network 106 is used by one or more computing or data storage devices (e.g., one or more databases 110 or other computing units described herein) for implementing the medical device data platform 102 and other services, applications, or modules in the network environment 100.

In one implementation, the network environment 100 includes at least one server 112 hosting a website or an application that the user may visit to access the medical device data platform 102 and/or other network components, including device programming machine that reside in a physician office and are utilized when in the presence of a patient. The server 112 may be a single server, a plurality of servers with each such server being a physical server or a virtual machine, or a collection of both physical servers and virtual machines. In another implementation, a cloud hosts one or more components of the network environment 100. The user devices 108, the server 112, and other resources connected to the network 106 may access one or more other servers to access to one or more websites, applications, web services interfaces, storage devices, computing devices, or the like that are used for integrated healthcare delivery. The server 112 may also host a search engine that the medical device data platform 102 uses for accessing, searching for, and modifying patient data, team member data, education data, and other data. In one implementation, the medical device data platform 102 provides access to data and/or other information of one or more medical devices 104.

Medical devices 104 may be in communication with network 106 to provide operational data to the medical device data platform 102. For example, cardiac implantable electronic devices (CIED), such as implantable cardioverter defibrillators (ICDs) and the like, often transmit information relating to the operation of the device outside the body for analysis, programming, and/or the like. In some cases, a clinic retrieves data for the medical devices 104 from a device manufacturer secure website. The data generated by the medical devices 104 is typically reviewed by a provider.

A typical medical care provider monitors transmissions from the full range of manufacturers and device models and types. Providers rarely implant and monitor a single device type. In terms of data transmissions and modalities of review of data transmissions, each of the manufacturers uses unique and proprietary data formats, displays, access protocols, reports, and programming machines. With the medical care provider receiving data in this wide range of unique and disparate formats, it is challenging for the medical care provider to efficiently manage the care of patients. The burdens of data access and management are a major impediment to better patient care. Cumbersome workflow creates excessive costs, impedes adoption of remote care, which is a superior care modality, leads to missed reimbursement opportunities, and forces some clinics to abandon remote care and instead push patients to the lower volume of in-office care, which leads to even further care reduction due to low patient compliance and greater healthcare system burdens associated with increased hospitalization. As such, as described in more detail below, the medical device data platform 102 thus allows a user to manage, analyze, and/or store data from the one or more medical devices 104 across various device types and disparate manufacturers.

FIG. 2 is a block diagram of a medical device data communication system 200 including an example medical device data platform 202. As depicted in FIG. 2, the medical device data platform 202 includes an integration manager 204, an actionability evaluator 206, an image processor 208 and a language processor 210.

In one implementation, the medical device data platform 202 is in communication with multiple implantable medical device manufacturer systems 220 and multiple clinic systems 230. The medical device data platform 202 may be communicatively coupled with the manufacturer systems 220 and the clinic systems 230 via a wide area network (WAN), such as, for example, the Internet. In some implementations, each of the manufacturer systems 220 may be associated with a unique medical device manufacturer. In other examples, each of the manufacturer systems 220 may be associated with a unique pairing of an medical device manufacturer and a particular medical clinic or office. In the example of the system 200 of FIG. 2, the manufacturer system 220 may include a manufacturer platform 222, such as, for example, a web server, that retrieves from a manufacturer database 224 diagnostic and related data previously uploaded from a plurality of medical devices. Such data may have been retrieved previously from the various medical devices by way of a clinic or office, or by way of a remote monitor, as described above.

The integration manager 204 accesses the data obtained by the medical devices from each of the manufacturer systems 220. In an example, the integration manager 204 may provide a clinical information system (CIS) identifier associated with a particular clinic to the manufacturer platform 222 to retrieve the diagnostic data and related information. In some implementations, messages between the integration manager 204 and the manufacturer platform 222 may be in the form of alternative, enhanced, or augmented data messages. In one example, IDCO messages often contain information formatted as summary reports in Portable Document Format (PDF). In another example, IDCO-like messages may provide more detailed or “raw” data, such as numerical and/or graphical electrogram (EGM) data regarding arrhythmia or other cardiac episodes detected by the implantable device in integer, floating-point, or another data format. Such information may facilitate easier and/or more detailed processing of the device data within the medical device data platform 202.

The medical device data platform 202 may then process the retrieved information to generate patient-oriented information and/or provider-oriented information. In some examples, the retrieved information from the various medical devices may be processed into a format that is unified or generalized across all manufacturers and/or devices of a particular type.

The integration manager 204 may forward the device data to the various other components of the medical device data platform 202 including actionability evaluator 206, which may then further process the data. The actionability evaluator 206 provides automated sorting or filtering of the retrieved device data to lessen the burden on the clinic staff and medical care providers by identifying data which does not involve immediate action. As described above, the data may be labeled as actionable or non-actionable. As discussed herein, non-actionable data, or non-clinical data, refers to any data received which does not trigger any further action. Non-actionable data may trigger or otherwise involve routine follow-ups, but in general, does not involve any immediate action by the clinic staff or the medical care provider. By filtering out, or otherwise labelling non-actionable data, the actionable data becomes more available and accessible to the clinic staff and the medical care providers. The actionability evaluator 206 processes the retrieved device data to determine if any of the data represents actionable data. Similarly, the lack of any identification of actionable data can result in a determination that the data is non-actionable.

The image processor 208 may be configured to classify one or more images taken from the received data (e.g. EGM related data) from the plurality of medical devices 104 as actionable or non-actionable. The image processor 208 may analyze the one or more images using image analysis algorithms, such as deep learning algorithms, convolutional neural networks, and/or the like. Based on the health condition identified, the image processor 208 assigns an appropriate category to each analyzed image. Additionally, or alternatively, the image processor 208 may perform image classification or image analysis of images obtained from the manufacturer, for example collected using an application programming interface from the manufacturer's website or other data storages.

The language processor 210 may analyze the retrieved information, particularly the portions of the data which represent text, to determine actionable items contained therein. The language processor 210 may use natural language processing (NLP) techniques using to identify specific keywords or text that indicate data or events requiring attention of the medical care provider.

In an implementation, when the image analysis algorithm identifies a health condition within an image that meets the predefined actionable criteria, it assigns an appropriate label to that image reflecting its clinical significance. A representative sample can be compared against the human labels attached to the data to measure accuracy of the image classification module.

As illustrated in FIG. 2, a user (e.g., one or more doctors, nurses, technicians, analysts, etc.) may employ a clinic system 230 to retrieve the processed device information, possibly including the analytics and other advanced information of one or more medical devices. In some examples, the clinic system 230 may include a clinic access system 232 (e.g., a desktop computer, a laptop computer, a tablet computer, a smart phone, etc.) from which the user may access a web portal to retrieve provider-oriented information, such as the medical device data platform 102. As shown in FIG. 2, the clinic system 230 may also include one or more of an electronic medical records manager system 234 configured to store and facilitate access to medical records of the patients of the clinic, and a health information exchange (HIE) system 236 configured to exchange health and medical information for the patients of the clinic with other computing systems external to the clinic system 230.

In some examples, the integration manager 204 may retrieve the device diagnostic data and other device information via a communication connection with one or more of the medical devices, thus possibly reducing the need for one or more of the manufacturer systems 220. In those examples and others, the integration manager 204 may forward or “push” the unprocessed and/or processed device information, as well as the analytics and other advanced information to one or more of the manufacturer systems 220 for use by the corresponding manufacturers of the devices.

The clinic access system 232 may be configured to present a web interface to the clinic staff including the physician to provide the provider-oriented device data information corresponding to the patients of that clinic. The clinic access system 232 may prompt a log on of the clinic staff to allow access to the provider-oriented information, as appropriate. In some examples, logging on to clinic access system 232 may facilitate access by clinic staff to other systems external to or located within the clinic system. In addition, the clinic access system 232 may provide an application programming interface (API) that facilitates provider access to electronic health records (EHRs) of the patient that may contain access points, such as, for example, embedded web links, to the device-related information.

FIG. 3 depicts a flowchart depicting the analysis 300 of a data transmission 302. In this example, the analysis consists of four separate categories with which each data transmission may be evaluated within. Each of the categories includes different parameters and thresholds for what is considered actionable. In some instances, other categories may be included as necessary. Each category may involve a different analysis and the determination is one of actionable or non-actionable. As depicted in FIG. 3, the categories include: device events 304, device measurements 306, image based data 308, and text based data 310. The transmission 302 is evaluated based upon each of these categories. The evaluation in any one of these categories may result in a determination that the transmission as a whole includes actionable data that should be brought to the medical care provider's attention. If the evaluation does not result in the identification of any actionable data, the transmission can be labeled as non-actionable.

Within the device events 304 category, transmissions including recorded events may be flagged as actionable. If there are any events detected within the transmission, the medical care provider will likely want to follow up and/or review the event. Further, in some instances, the medical device data platform 202 may further analyze each of the events and assign a level of urgency to the event. In this instance, the determination as to whether the transmission is actionable may also be based on the assigned level of urgency.

Within the device measurements 306 category, data relating to device measurements can identify patterns that define behavior of the medical device that deviates from normal device functioning. One example could include comparing measurements in current transmissions against previous transmissions to observe any extreme changes. For example, a threshold can be set for an acceptable level of percentage change in a given measurement from one transmission to the next. In some example, the threshold may be an upper limit or a lower limit for the particular measurement. In another example, the threshold may relate to a percentage of change, such that, if the percentage of change of the measurement exceeds the threshold, the transmission is labeled as actionable. The device measurements 306 category may also monitor for issues with battery or other functions of the device itself.

The image based data 308 category is particularly relevant to cardiac devices. As discussed above, cardiac devices may be manufactured by many different manufacturers. However, regardless of the manufacturer, reports from the cardiac devices often include segments of heart rhythm data, or rhythm strips. These rhythm strips can be analyzed to determine if there are any potential issues to be reviewed by a medical care provider. In this instance, the image processor 208 of the medical device data platform 202 can be utilized to isolate and analyze the rhythm strip. The medical device data platform 202, and specifically the image processor 208, parses the rhythm strip from the received report. The image processor 208 includes machine learning capabilities used to analyze and classify the extracted rhythm strip. For example, the image processor 208 may utilize a machine learning algorithm, such as convolutional neural network trained on previously received rhythm data that has been interpreted or classified by medical care providers. The image processor 208 may convert the image into discrete data that can be further processed by the machine learning algorithm. The machine learning algorithm is capable of receiving the rhythm strip data and determine whether the associated transmission includes any abnormal data which represents an actionable item.

The text based data 310 category involves analyzing the text, such as clinical notes, included within the report provided by the manufacturer to determine whether the transmission is actionable. For example, certain words or phrases may indicate higher priority or severity, such that the medical care provider may want to review. The language processor 210 of the medical device data platform may be utilized to parse and analyze the clinical notes. In some instances, the language processor may utilize a machine learning algorithm, such as natural language processing (NLP) to interpret the clinical notes and make a determination regarding whether or not the transmission is actionable.

FIG. 4 is a flow diagram of an example method 400 of providing provider-oriented information based on data transmissions received from a plurality of medical devices. In some examples, the method 400 is performed using the medical device data platform of FIG. 1. However, other systems and/or devices may be employed in executing the method 400 in other examples.

The method 400 includes obtaining 402 a plurality of data transmission from a plurality of medical devices associated with a plurality of patients. The method 400 further includes generating 404 provider-oriented information by applying a label to each data transmission of the plurality of data transmissions, wherein the label is one of an actionable label or a non-actionable label. The method 400 further includes generating 406 a provider portal displaying the plurality of data transmissions and the applied actionable label associated with each data transmission of the plurality of data transmissions.

While the operations 402 through 406 are shown as being performed in an example order, other orders of performance for the operations 402 through 406 are also possible.

The resulting medical device data access system implementations disclosed herein may be advantageous for several reasons. For example, the process described above of obtaining data stored within medical devices, which number in the millions globally, may be automatically facilitated across all manufacturers, clinics, and patients, thus reducing the need for additional IT personnel to be involved with the periodic uploading and analysis of device data, and improving efficiency. Additionally, medical device data received from different medical devices is filtered to highlight device data that necessitates emergency attention by a medical care provider, and thereby helping in expediting the patient care. In some implementations, the processed data may be integrated with existing patient EHRs or EMRs to facilitate simplified access to that data by the patient or care provider.

Additionally, the medical device data access system may standardize care across multiple systems or personnel, and, thereby, solves vulnerabilities due to a human error or subjective conclusion about a relevant event leading to an improper outcome. Further, as more data becomes available over time, the machine learning based model becomes more accurate over time, and more accurate and relevant data is available for medical care providers.

Referring to FIG. 5, a detailed description of an example computer system 500 having one or more computing units that may implement various systems and methods discussed herein is provided. The computer system 500 may be applicable to the medical device data platform 102 and/or the user computing device 108 and other computing or network devices. It will be appreciated that specific implementations of these devices may be of differing possible specific computing architectures not all of which are specifically discussed herein but will be understood by those of ordinary skill in the art.

The computer system 500 may be a computing system is capable of executing a computer program product to execute a computer process. Data and program files may be input to the computer system 500, which reads the files and executes the programs therein. Some of the elements of the computer system 500 are shown in FIG. 5, including one or more hardware processors 502, one or more data storage devices 504, one or more memory devices 506, and/or one or more ports 508, 510. Additionally, other elements that will be recognized by those skilled in the art may be included in the computer system 500 but are not explicitly depicted in FIG. 5 or discussed further herein. Various elements of the computer system 500 may communicate with one another by way of one or more communication buses, point-to-point communication paths, or other communication means not explicitly depicted in FIG. 5.

The processor 502 may include, for example, a central processing unit (CPU), a microprocessor, a microcontroller, a digital signal processor (DSP), and/or one or more internal levels of cache. There may be one or more processors 502, such that the processor 502 comprises a single central-processing unit, or a plurality of processing units capable of executing instructions and performing operations in parallel with each other, commonly referred to as a parallel processing environment.

The computer system 500 may be a conventional computer, a distributed computer, or any other type of computer, such as one or more external computers made available via a cloud computing architecture. The presently described technology is optionally implemented in software stored on the data stored device(s) 504, stored on the memory device(s) 506, and/or communicated via one or more of the ports 508, 510, thereby transforming the computer system 500 in FIG. 5 to a special purpose machine for implementing the operations described herein. Examples of the computer system 500 include personal computers, terminals, workstations, mobile phones, tablets, laptops, personal computers, multimedia consoles, gaming consoles, set top boxes, and the like.

The one or more data storage devices 504 may include any non-volatile data storage device capable of storing data generated or employed within the computer system 500, such as computer executable instructions for performing a computer process, which may include instructions of both application programs and an operating system (OS) that manages the various components of the computer system 500. The data storage devices 504 may include, without limitation, magnetic disk drives, optical disk drives, solid state drives (SSDs), flash drives, and the like. The data storage devices 504 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1806 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 504 and/or the memory devices 506, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 500 includes one or more ports, such as an input/output (I/O) port 508 and a communication port 510, for communicating with other computing, network, or vehicle devices. It will be appreciated that the ports 508, 510 may be combined or separate and that more or fewer ports may be included in the computer system 500.

The I/O port 508 may be connected to an I/O device, or other device, by which information is input to or output from the computer system 500. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computer system 500 via the I/O port 508. Similarly, the output devices may convert electrical signals received from computer system 500 via the I/O port 508 into signals that may be sensed as output by a human, such as sound, light, and/or touch. The input device may be an alphanumeric input device, including alphanumeric and other keys for communicating information and/or command selections to the processor 502 via the I/O port 508. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computer system 500 via the I/O port 508. For example, an electrical signal generated within the computer system 500 may be converted to another type of signal, and/or vice-versa. In one implementation, the environment transducer devices sense characteristics or aspects of an environment local to or remote from the computer system 500, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computer system 500, such as, physical movement of some object (e.g., a mechanical actuator), heating or cooling of a substance, adding a chemical substance, and/or the like.

In one implementation, a communication port 510 is connected to a network by way of which the computer system 500 may receive network data useful in executing the methods and systems set out herein as well as transmitting information and network configuration changes determined thereby. Stated differently, the communication port 510 connects the computer system 500 to one or more communication interface devices configured to transmit and/or receive information between the computer system 500 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 510 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 510 may communicate with an antenna or other link for electromagnetic signal transmission and/or reception.

The system set forth in FIG. 5 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

Implementations disclosed herein include various operations that are described in this specification. As discussed above, the operations may be performed by hardware components and/or may be embodied in machine-executable instructions, which may be used to cause a general-purpose or special-purpose processor programmed with the instructions to perform the operations. Alternatively, the operations may be performed by a combination of hardware, software, and/or firmware.

The performance of one or more operations described herein may be distributed among one or more processors, not only residing within a single machine, but deployed across a number of machines. In some examples, the one or more processors or processor-implemented modules may be located in a single geographic location (e.g., within a home environment, an office environment, or a server farm). In other implementations, the one or more processors or processor-implemented modules may be distributed across a number of geographic locations.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores may be arbitrary, and particular operations are illustrated in a context of specific illustrative configurations. In general, structures and functionality presented as separate resources in the example configurations may be implemented as a combined structure or resource. Similarly, structures and functionality presented as a single resource may be implemented as separate resources.

While the present disclosure has been described with reference to various implementations, these implementations are illustrative, and the scope of the disclosure is not limited to such implementations. Various modifications and additions can be made to the exemplary implementations discussed herein without departing from the scope of the disclosure. For example, while the implementations described above refer to particular features, the scope of this disclosure also includes implementations having different combinations of features, as well as implementations that do not include all of the described features. Accordingly, the scope of the disclosure is intended to embrace all such alternatives, modifications, and variations, together with all equivalents thereof.

1. A medical device data platform comprising:
an integration manager configured to obtain a plurality of data transmissions wirelessly from a plurality of medical devices via a network, each data transmission originating from a corresponding medical device associated with a patient, each data transmission including at least one of image data or text data; and
an actionability evaluator having at least one processor classifying each data transmission of the plurality of data transmissions as one of either actionable or non-actionable based on a care protocol of the patient associated with the corresponding medical device, the actionability evaluator applying an actionable label to each data transmission of the plurality of data transmissions determined to be actionable and a non-actionable label to each data transmission of the plurality of data transmissions determined to be non-actionable, the actionability evaluator generating provider-oriented information corresponding to a subset of the plurality of data transmissions with the actionable label. label to the data transmission,

2. The medical device data platform of claim 1, wherein the actionability evaluator identifies at least a portion of one of the plurality of data transmissions as being related to an event and applies the actionable label to the data transmission based on an analysis of the event.

3. The medical device data platform of claim 1, the actionability evaluator identifying one or more device measurements or changes in measurements within one of the plurality of data transmissions, comparing the one or more device measurements or changes in measurements to a threshold associated with the one or more device measurements or changes in measurement, and upon determining that the one or more device measurements or changes in measurements exceeds the threshold, applying the actionable label to the data transmission.

4. The medical device data platform of claim 1 further comprising:
an image processor isolating an image included within one of the plurality of data transmissions, identifying that the image includes an abnormality, and applying the actionable label to the data transmission based on the abnormality.

5. The medical device data platform of claim 4, wherein the image processor utilizes a convolutional neural network to identify the abnormality.

6. The medical device data platform of claim 1, further comprising:
a language processor isolating clinical notes included within one or more of the plurality of data transmissions, determining that the clinical notes indicate a potential issue for review by a medical care provider, and applying the actionable label to the data transmission based on the potential issue.

7. The medical device data platform of claim 6, wherein the language processor utilizes a natural language processing (NLP) algorithm to identify the potential issue.

8. The medical device data platform of claim 1, wherein the non-actionable label indicates a low priority of an associated data transmission.

9. The medical device data platform of claim 1, wherein generating the provider-oriented information includes combining one or more of the plurality of data transmissions with data associated with a corresponding patient from an electronic medical record system to create an enriched data set.

10. The medical device data platform of claim 1, wherein the subset is a first subset, the medical device data platform further comprising:
a provider portal displaying the first subset of the data transmissions with the actionable label while filtering out a second subset of data transmissions with the non-actionable label.

11. A method comprising:
obtaining a plurality of data transmissions from a plurality of medical devices associated with a plurality of patients, each data transmission including a summary report generated by a manufacturer of a corresponding medical device from which the data transmission originated;
classifying each data transmission of the plurality of data transmissions as one of either actionable or non-actionable based on a set of rules corresponding to a patient associated with a corresponding medical device;
applying an actionable label to each data transmission of the plurality of data transmissions determined to be actionable and a non-actionable label to each transmission of the plurality of data transmissions determined to be non-actionable;
generating provider-oriented information corresponding to a subset of the plurality of data transmissions with the actionable label; and
generating a provider portal displaying the subset of the data transmissions with the actionable label.

12. The method of claim 11, further comprising:
identifying at least a portion of one of the plurality of data transmissions as being related to an event; and
applying the actionable label to the data transmission based on an analysis of the event.

13. The method of claim 11, further comprising:
identifying one or more device measurements or changes in measurements within one of the plurality of data transmissions;
comparing the one or more device measurements or changes in measurements to a threshold associated with the one or more device measurements or changes in measurements; and
upon determining that the one or more device measurements or changes in measurements exceed the threshold, applying the actionable label to the data transmission.

14. The method of claim 11, further comprising:
isolating an image included within one of the plurality of data transmissions;
identifying that the image includes an abnormality; and
applying the actionable label to the data transmission based on the abnormality.

15. The method of claim 14, wherein a convolutional neural network is utilized to identify the abnormality.

16. The method of claim 11, further comprising:
isolating clinical notes included within one of the plurality of data transmissions;
determining that the clinical notes indicate a potential issue for review by a medical care provider; and
applying the actionable label to the data transmission based on the potential issue.

17. The method of claim 16, wherein a natural language processing (NLP) algorithm is utilized to determine the potential issue.

18. The method of claim 11, wherein the non-actionable label indicates a low priority of an associated data transmission.

19. The method of claim 11, wherein generating the provider-oriented information includes combining one or more of the plurality of data transmissions with data associated with a corresponding patient from an electronic medical record system to create an enriched data set.

20. (canceled)

21. A method comprising:
obtaining a plurality of data transmissions from a plurality of medical devices associated with a plurality of patients, each data transmission including at least one of image data or text data;
classifying each data transmission of the plurality of data transmissions as one of either actionable or non-actionable based on a care protocol of the patient associated with a corresponding medical device of the plurality of medical devices;
applying an actionable label to each data transmission of the plurality of data transmissions determined to be actionable and a non-actionable label to each transmission of the plurality of data transmissions determined to be non-actionable;
generating provider-oriented for the plurality of data transmissions corresponding to a subset of the plurality of data transmissions with the actionable label; and
generating a provider portal displaying the subset of data transmissions with the actionable label.
